(19) United States
(12) Patent Application Publication
(10) Pub. No.: US 2002/0182223 A1
(43) Pub. Date: Dec. 5, 2002

(54) Method of rapidly generating double-stranded RNA and methods of use thereof

(76) Inventors: Douglas J. LaCount, Iowa City, IA (US); John E. Donelson, Iowa City, IA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/870,407

(22) Filed: May 30, 2001

Related U.S. Application Data
(60) Provisional application No. 60/209,885, filed on Jun. 2, 2000.

(51) Int. Cl. A61K 39/002; C12N 15/74; C12N 1/10

(52) U.S. Cl. 424/191.1; 435/320.1; 435/258.1

(57) ABSTRACT

The present invention provides eukaryotic double-stranded RNA (dsRNA) expression vectors containing two promoters directed head-to-head with a designated intervening sequence of interest that is effective in eukaryotic cells containing the dsRNA expression vector, and a vaccine using an attenuated eukaryotic pathogenic cell. The present invention also provides methods of vaccinating against colonization or infection of a eukaryotic pathogen. The present invention further provides methods of generating double-stranded RNA (dsRNA) and screening designated nucleic acids capable of inhibiting expression of an essential eukaryotic gene.

### CLAIM OF PRIORITY

This non-provisional application claims priority under 35 U.S.C. 119 from U.S. Provisional Application Serial No. 60/209,885, filed Jun. 2, 2000, which application is incorporated herein by reference.

The invention was made with the support of NIH Grant No. AI40591. The U.S. government has certain rights in the invention.

### BACKGROUND OF THE INVENTION

Several communicable diseases are caused by protists, such as protozoan parasites. Examples of these diseases are African sleeping sickness, Chagas disease, leishmaniases, toxoplasmosis and malaria. The treatments for these diseases, which are common in certain regions of the world, are quite expensive, prolonged and often involve the use of toxic drugs. Therefore, these types of diseases have a serious impact on the infected communities.

African Sleeping Sickness

African sleeping sickness is caused by protozoan parasites of the genus Trypanosoma. The parasite is transmitted to humans through the bite of the tsetse fly of the genus Glossina. There are two forms of the human disease, each caused by a different parasite: Trypanosoma brucei gambiense, which causes a chronic infection lasting years and affecting countries of western and central Africa; and Trypanosoma brucei rhodesiense, which causes acute illness lasting several weeks in countries of eastern and southern Africa. When a person becomes infected, the trypanosome multiplies in the blood and lymph glands, crossing the blood-brain barrier to invade the central nervous system where it causes major neurological disorders. Infection by trypanosomes causes neurological alterations that are often irreversible, even after successful treatment. Psychomotor and neurological retardation, even among cured children, is frequent. Without treatment, the disease is invariably fatal.

Sleeping sickness is a daily threat to more than 60 million people in 36 countries of sub-Saharan Africa, 22 of which are among the least developed countries in the world. However, only 3 to 4 million of these people are under surveillance and the 45,000 cases reported in 1999 do not reflect the reality of the situation, but simply show the absence of case detection. The estimated number of people thought to have the disease is between 300,000 and 500,000.

Detection of people infected with sleeping sickness and subsequent patient treatment require well-trained staff, resources, drugs and well-equipped health centers. Furthermore, without systematic screening of exposed populations and without treatment, the majority of sick people will die without ever having been diagnosed.

Sleeping sickness has a major impact on the development of rural areas by decreasing the labor force and hampering production and work capacity. It remains a major obstacle to the development of entire regions. In countries such as Angola, Democratic Republic of Congo or Sudan, the operational capacity to respond to the epidemic situation is largely surpassed and in certain endemic areas the observed prevalence is huge. In numerous provinces in these countries, a prevalence greater than 20% has been reported.

Chagas Disease

Chagas disease, named after the Brazilian physician Carlos Chagas who first described it in 1909, exists only on the American Continents. It is caused by a flagellate protozoan parasite, Trypanosoma cruzi, which is transmitted to humans by triatomine insects known popularly in the different countries as “kissing bugs”, “vinchuca”, “barbeiro”, “chipo” etc. The geographical distribution of the human T. cruzi infection extends from Mexico to the south of Argentina. The disease affects 16-18 million people and some 100 million, i.e., about 25% of the population of Latin America, is at risk of acquiring Chagas disease.

There are two stages of the human disease: the acute stage, which appears shortly after the infection, and the chronic stage, which appears after a silent period that may last many years. The lesions of the chronic phase irreversibly affect internal organs such as the heart, esophagus and colon and the peripheral nervous system. After several years of an asymptomatic period, 27% of those infected develop cardiac symptoms, which may lead to sudden death, 6% develop digestive damage, mainly megaviscera, and 3% will present peripheral nervous involvement.

The risk of infection with Chagas disease is directly related to poverty. The blood-sucking triatomine bug that transmits the parasite finds a favorable habitat in crevices in the walls and roofs of poor houses in rural areas and in the peripheral urban slums. The rural/urban migration movements that occurred in Latin America in the 1970's and 1980's changed the traditional epidemiological pattern of Chagas disease and transformed it into an urban infection that can be transmitted by blood transfusion. The figures of infection of blood in blood banks in some selected cities of the continent vary between 3.0 and 53.0%, thus showing that the prevalence of T. cruzi-infected blood can be higher than that of HIV infection and Hepatitis B and C.

According to the UNDP Human Development Report the estimated average annual per-capita gross domestic product in Latin America is US$ 2,966. Thus, the economic loss for the Continent due to early mortality and disability by this disease in economically most productive young adults currently amounts to US$ 8,156 millions, which is equivalent to 2.5% of the external debt of Latin America in 1995.

Leishmaniases and Leishmania/HIV Co-infections

The leishmaniases are a globally widespread group of parasitic diseases with a broad range of clinical manifestations. They are caused by several species belonging to the genus Leishmania, a flagellate protozoa that is transmitted exclusively by the bite of the female phlebotomine sandfly. The sandfly becomes infected when taking a blood meal from a reservoir host, including humans as well as wild and domestic animals. Most leishmaniases are zoonotic (transmitted to humans from animals), and humans become infected only when accidentally exposed to the natural transmission cycle. However, in the anthroponotic forms (those transmitted from human to human through the sandfly vector), humans are probably the sole reservoir host.

Since 1993, regions that are Leishmania-endemic have spread significantly, accompanied by a sharp increase in the number of recorded cases of the disease. The geographic spread is due to factors related mostly to development, including massive rural-urban migration, agro-industrial projects that bring non-immune urban dwellers into endemic rural areas, and man-made environmental changes like dams, irrigation systems and wells. AIDS and other immunosuppressive conditions increase the risk of Leishmania-infected people developing visceral illness, and in certain areas of the world the risk of co-infection with HIV is rising due to epidemiological changes.

Visceral leishmaniases (VL), also known as kala azar, is the most severe form of the disease, with an almost 100% mortality rate if left untreated. It is characterized by irregular bouts of fever, substantial weight loss, swelling of the spleen and liver, and anemia. Mucocutaneous leishmaniases, or espundia, produces lesions, which can lead to extensive and disfiguring destruction of mucous membranes of the nose, mouth and throat cavities. Cutaneous leishmaniases (CL) can produce large numbers of skin ulcers—as many as 200 in some cases—on the exposed parts of the body such as the face, arms and legs, causing serious disability and leaving the patient permanently scarred. Diffuse cutaneous leishmaniasis (DCL) never heals spontaneously and tends to relapse after treatment.

The leishmaniases are now endemic in 88 countries on five continents—Africa, Asia, Europe, North America and South America—with a total of 350 million people at risk. Case notification is compulsory in only 40 endemic countries, and the World Health Organization (WHO) is strongly encouraging the remaining 48 endemic countries to follow suit. Currently, it is believed that 12 million cases of all forms of the disease exist worldwide. Of the 2 million new cases of leishmaniases estimated to occur annually, only 600,000 are officially declared. Of the 500,000 new cases of visceral leishmaniases that occur annually, 90% are in five countries—Bangladesh, Brazil, India, Nepal and Sudan. 90% of all cases of mucocutaneous leishmaniases occur in Bolivia, Brazil and Peru. 90% of all cases of cutaneous leishmaniases occur in Afghanistan, Brazil, Iran, Peru, Saudi Arabia and Syria, with 1-1.5 million new cases reported annually worldwide.

The overlapping geographical distribution of visceral leishmaniases (VL) and AIDS is increasing, due to two main factors: the spread of the AIDS pandemic in suburban and rural areas of the world, and the simultaneous spread of VL into several new areas as a result of epidemiological changes arising from urbanization. Cases of Leishmania/HIV co-infections are being reported more frequently in various parts of the world, with important clinical, diagnostic, chemotherapeutic, epidemiological and economic implications.

Although people are often bitten by sandflies infected with Leishmania protozoa, most do not develop the disease. However, among persons who are immunosuppressed (i.e., HIV positivity, immunosuppressive treatment for organ transplants, hematological malignancy, auto-immune diseases), cases quickly evolve to a full clinical presentation of the disease. Leishmania/HIV co-infections are considered a real threat, especially in southern Europe. Of the first 700 cases of co-infection that have been reported to the WHO, 673 cases were from the region: Spain (413 cases), Italy (130 cases), France (127 cases), and Portugal (3 cases). Of these, 85.7% were males, 85.7% were young adults (20-40 years old), and 71% were intravenous drug users.

In southern Europe, VL is now the most common opportunistic parasitic infection among HIV-positive persons. The mean survival of co-infected patients is only 13 months. They are severely immunosuppressed, and after the first course of chemotherapy, relapses are much more frequent (52%) for co-infections than for patients suffering only from leishmaniases. From 25% to 70% of adults with VL are also HIV-positive, and 1.5% to 9% of AIDS cases suffer from newly acquired or reactivated VL.

Most co-infections in the Americas are reported from Brazil, where the incidence of AIDS has risen from 4.3 cases per 100,000 inhabitants in 1986 to 18.4 in 1994. As HIV transmission has spread into rural areas, VL has simultaneously become more urbanized—especially in northeastern Brazil—increasing the risk of overlapping infection.

In Eastern Africa, cases of Leishmania/HIV co-infections have been reported in Ethiopia (27), Kenya (13), Malawi (1) and Sudan (3). The risk of overlap is increasing due to a number of factors: mass migration, civil unrest or war, resettlement programmes, and promiscuity and prostitution in refugee camps. In North Africa, a few cases have been reported in Algeria, Morocco and Tunisia and in Western Africa one case each in Cameroon and Guinea Bissau. It should be emphasized that these figures are based upon random reports only.

Leishmania/HIV co-infections impose specific difficulties in terms of diagnosis and treatment. The usual clinical features (fever, weight loss, liver and spleen enlargement, inflammation of the lymph nodes) are not always present. The clinical diagnosis can also be made difficult by associated diseases such as cryptosporidium, cryptococcosis, cytomegalovirus infection or mycobacterial infection. The serological diagnosis is frequently negative due to a 20-40% reduction of sensitivity of the tests. HIV-positive patients have difficulty in producing antibodies against new infection agents, especially at a late stage or during relapses. Consequently, there is a need to combine two or more techniques.

Although multiple visceral localizations are frequent (blood, normal skin, digestive tract, lungs, CNS), parasitological diagnosis can be difficult and has to be repeated to orient the treatment. Bone marrow aspirate (BMA) remains the safest and most sensitive technique, but spleen aspirate and liver biopsy are also used. When BMA cannot be performed, a search for Leishmania can be conducted in peripheral blood samples.

Treatment for co-infected patients is aimed at clinical and parasitological cures and prevention of relapses. Unfortunately, in such patients treatment failure and relapses due to drug resistance and drug toxicity are very common. In southern Europe, follow-up studies using pentavalent antimonials show a positive response in 83% of cases. However, 52% of patients relapse within a period of one month to three years, with the number of relapses ranging from one to four. The main alternative drugs include pertamidine, amphotericin B and amphotericin B encapsulated in liposomes. This encapsulation reduces the occurrence of side effects, but relapses still occur and the drug remains extremely expensive.

Leishmania/HIV co-infections can lead to epidemiological changes, which modify the traditional patterns of zoonotic VL. Co-infected patients harbor a high number of Leishmania in their blood, and so there is also a risk of them becoming reservoirs of the disease (that is, infective for the sandfly vector) as in anthroponotic foci in Bangladesh, India, Nepal and East Africa, and hence increasing the risk of future epidemics.

Experimentally, sandflies can be infected through a bloodmeal using a very low quantity of blood from co-infected patients. The quantity may be lower than the content of a needle. As 71% of co-infected patients in southern Europe are intravenous drug users, transmission of Leishmania may be possible through the sharing of syringes in this population group.

Toxoplasmosis

Toxoplasma gondii is a protozoan parasite that infects most species of warm-blooded animals, including humans, and can cause the disease toxoplasmosis. The definitive host for T. gondii is the house cat and other members of the Felidae family. Human infection may be acquired by (1) ingestion of undercooked infected meat containing Toxoplasma cysts; (2) ingestion of the oocyst from fecally contaminated hands or food; (3) organ transplantation or blood transfusion, (4) transplacental transmission, and (5) accidental inoculation of the tachyzoite form of the parasite. The parasites form tissue cysts, most commonly in skeletal muscle, myocardium, and brain; these cysts may remain throughout the life of the host. About 13% of the world's population has been exposed to Toxoplasma, as determined by serology, and most infected persons do not develop the disease. However, the disease prevalence varies greatly among populations depending on (1) the diet, i.e., if uncooked or poorly cooked meat is eaten, and (2) the existence of immuno-compromised persons, i.e., if a patient is undergoing suppressive immunotherapy as in the case of transplant patients, or if the patient is suffering from HIV. In addition, newly born infants are particularly susceptible to heavy infections.

The disease is generally classified as being (1) acute, (2) sub-acute and (3) chronic. The most common symptoms of acute infection are swollen lymph glands, associated fever headaches and anemia, and are often mistaken for flu symptoms. The sub-acute form of the disease is marked by continuing dispersal of the tachyzoite form of the parasite to other tissues such as heart, liver, brain and eyes with accompanying cellular destruction. In chronic infections, the host mammal's immunity builds up, suppressing tachyzoite proliferation and leading to cyst formation. The cysts can remain dormant and intact for years until for unknown reasons, the host's immune response is depressed, perhaps as a result an other pathogenic infection or chemically induced immune suppression. Under these conditions the cysts burst and the bradyzoites are released to reinfect other tissues.

One of the most serious results of Toxoplasma infections is congenital toxoplasmosis, which occurs when a mother contracts the infection during pregnancy. The organism can cause abortion of the fetus or can cause severe malformation of the fetus. If the woman is already infected prior to conception, there is little danger. The greatest risk of congenital toxoplasmosis occurs during the first trimester of pregnancy. However, it is during the third trimester that the highest level of transmission occurs. This is thought to be related to the much larger size of the uterus. Toxoplasma infections are also a problem in mammals other than humans. Sheep are also particularly susceptible, with congenital toxoplasmosis accounting for over half the ovine abortions in both England and New Zealand.

Malaria

Malaria is the world's most important tropical parasitic disease by far, and kills more people than any other communicable disease except tuberculosis. In many developing countries, and in Africa especially, malaria exacts an enormous toll in lives, in medical costs, and in days of labor lost. The causative agents in humans are four species of the Plasmodium protozoan (single-celled) parasite—P. falciparum, P. vivax, P. ovale mand P. malariae. Of these, P. falciparum and P. vivax account for about 95% of infections, and P. falciparum infections are the most lethal. The Plasmodium species are transmitted by female Anopheline mosquitoes, the number and type of which determine the extent of transmission in a given area. In addition, transmission is affected by climate and geography, and often coincides with the rainy season. Malaria is a curable disease if promptly diagnosed and adequately treated.

Malaria is a public health problem today in more than 90 countries, inhabited by a total of about 2.4 billion people—40% of the world's population. Worldwide prevalence of the disease is estimated to be about 300-500 million clinical cases each year. More than 90% of all malaria cases are in sub-Saharan Africa. Mortality due to malaria is estimated to be over 1 million deaths each year. The vast majority of deaths occur among young children in Africa, especially in remote rural areas with poor access to health services. Other high-risk groups are women during pregnancy, and non-immune travelers, refugees, displaced persons and laborers entering endemic areas. Malaria epidemics related to political upheavals, economic difficulties and environmental problems also contribute dramatically to death tolls and human suffering. Most malaria cases seen in the temperate zones of North America, Europe and Japan are in travelers recently returned from endemic areas and in immigrants from these areas.

Symptoms of malaria include fever, shivering, pain in the joints, headache, repeated vomiting, generalized convulsions and coma. Severe anemia (exacerbated by malaria) is often the attributable cause of death in areas with intense malaria transmission. If not treated, the disease, particularly that caused by P. falciparum, progresses to severe malaria, which is frequently associated with death.

Thus, there is an on-going need for both vaccines and more efficient, less toxic treatments for protist infections, such as protozoan parasites.

### SUMMARY OF THE INVENTION

The present invention provides a eukaryotic double-stranded RNA (dsRNA) expression vector effective in a eukaryotic cell having a designated DNA sequence of interest and a pair of promoters on opposite ends of the designated DNA, wherein the promoters are oriented towards each other and wherein each is capable of transcribing a strand of DNA into RNA. The eukaryotic cell may be a protist cell, which is a single-celled eukaryote. The protist may be a protozoan parasite cell, such as a Trypanosoma, Leishmania, Toxoplasma, or Plasmodia cell. In the dsRNA expression vector of the present invention, the designated DNA sequence of interest may be a random DNA sequence or a known DNA sequence. For example, the designated DNA sequence may be an essential gene from a protist. The designated DNA sequence may be a gene that is normally active during the protozoan's lifecycle when the protozoan is living in a mammalian host. It may not be active during the protozoan's lifecycle when the protozoan is living in an insect host.

Each of the pair of promoters in the expression vector may the same type of promoter, or they may be different promoters. At least one of the pair of promoters may be a eukaryotic promoter, such as a viral promoter, a ribosomal RNA promoter, a T. brucei variant surface glycoprotein (VSG) gene promoter, a procyclic acidic repetitive protein (PARP) gene promoter, or another unrelated eukaryotic promoter. One or both of the promoters may be a prokaryotic promoter, such as a bacteriophage T7 promoter, a bacteriophage T3 promoter, or bacteriophage SP6 promoter or another prokaryotic promoter. If a ribosomal RNA promoter or another unrelated eukaryotic promoter is used, it may be derived from Trypanosoma, Leishmania, Toxoplasma, Plasmodia, or another eukaryotic organism. The dsRNA expression vector of the present invention may use a vector backbone, and this backbone may be a Trypanosoma, Leishmania, Toxoplasma, or Plasmodia expression vector, or an expression vector specific for another eukaryotic organism.

The present invention also provides a eukaryotic cell containing a eukaryotic double-stranded RNA (dsRNA) expression vector having a designated DNA sequence of interest; and a pair of promoters on opposite ends of the designated DNA, wherein the promoters are oriented towards each other and wherein each is capable of transcribing a strand of DNA into RNA.

The present invention also provides a vaccine comprising a eukaryotic cell containing, in combination with a physiologically-acceptable, non-toxic vehicle, a eukaryotic double-stranded RNA (dsRNA) expression vector having a designated DNA sequence of interest and a pair of promoters on opposite ends of the designated DNA, wherein the promoters are oriented towards each other and wherein each is capable of transcribing a strand of DNA into RNA. The vaccine may further contain an immunological adjuvant. The eukaryotic cell of the vaccine may be a protist cell, such a protozoan parasite cell. For example, the protist cell may be a Trypanosoma, Leishmania, Toxoplasma, or Plasmodia cell.

The present invention further provides a method of protecting a susceptible mammal against colonization or infection of a eukaryotic pathogen by administering to the mammal an effective amount of a vaccine containing a eukaryotic pathogen cell, wherein the cell contains, in combination with a physiologically-acceptable, non-toxic vehicle, a eukaryotic double-stranded RNA (dsRNA) expression vector having a designated DNA sequence of interest and a pair of promoters on opposite ends of the designated DNA, wherein the promoters are oriented towards each other and wherein each is capable of transcribing a strand of DNA into RNA. The vaccine may further have an immunological adjuvant. The vaccine may be administered by subcutaneous or intramuscular injection, by oral ingestion, or be administered intranasally. The recipient mammal may be a human, dog, bovine, porcine, or equine.

The present invention further provides a method of generating double-stranded RNA (dsRNA) by culturing a eukaryotic cell that contains a eukaryotic double-stranded RNA (dsRNA) expression vector having a designated DNA sequence of interest; and a pair of promoters on opposite ends of the designated DNA, wherein the promoters are oriented towards each other and wherein each is capable of transcribing a strand of DNA into RNA.

The present invention also provides a method of screening designated nucleic acids capable of inhibiting expression of an essential eukaryotic gene by introducing a eukaryotic double-stranded RNA (dsRNA) expression vector into a eukaryotic cell, wherein the expression vector has a designated DNA sequence of interest and a pair of promoters on opposite ends of the designated DNA, wherein the promoters are oriented towards each other and wherein the expression vector produces dsRNA, and measuring the ability of the dsRNA to inhibit expression of the corresponding endogenous DNA.

### BRIEF DESCRIPTION OF THE DRAWINGS

FIG. 1. Generation of double-stranded RNA interference (dsRNAi) using two head-to-head promoters. A. Schematic diagram of the two-promoter plasmids p2rRNAprom and p2T7. The relative positions and orientations of the promoters are indicated by bent arrows. In both plasmids, the gene or sequence of interest is inserted between the two promoters. B-C. Differential interference contrast (DIC) images of wild-type YTAT 1.1 procyclic T. brucei cells 12 h after transfection of 2.5×10⁷ cells with 60 μg of p2rRNAprom or p2rRNAprom/αtub plasmid DNA, respectively. Panel C shows two wild-type cells and two FAT cells. D-E. DIC image of the T7 RNA polymerase-expressing cell line 29-13 prepared 12 h after transfection of 2.5×10⁷ cells with 40 μg of p2T7 or p2T7/αtub plasmid DNA, respectively. Scale bars indicate 10 μm.

FIG. 2. Inhibition of GFP expression with the two-T7 promoter vector. A. 82GFP cells, which constitutively express the T7 RNA polymerase and inducibly express GFP, were transfected with 40 μg of p2T7, p2T7/gfp, or p2T7/αtub plasmid DNA and immediately transferred to fresh media containing 1 μg ml⁻¹ tetracycline to induce GFP expression from the integrated copy of gfp. Merged DIC and fluorescent images were prepared 24 h after transfection. Scale bars indicate 10 μm. B. Analysis of GFP expression by flow cytometry following transfection. 82GFP cells were transfected with p2T7, p2T7/gfp, or pBS/gfp-AS (expresses antisense gfp from the T7 promoter) as described above and subjected to flow cytometry at 24 h after transfection. Dead cells were excluded by staining with propidium iodide. Fluorescent intensity is indicated on the x-axis as arbitrary fluorescent units.

FIG. 3. T. brucei fla1 knockout phenotypes. Differential interference contrast (DIC) images of T. brucei 29-13 cells (which express T7 RNA polymerase) transfected with 40 μg of p2T7 (A), p2T7/fla1 (B-G), or pBS/fla1-AS (fla1 antisense; H) plasmid DNA. Cells transfected with p2T7/fla1 exhibit a range of detached flagella phenotypes. B-C. cells with partially detached flagella; D. cells with partially or fully detached flagella, E. cell with fully detached flagellum; F. cell with one attached and one detached flagellum; G. cell with two detached flagella. Arrows indicate detached flagella. Scale bars (10 μm) are shown in the lower left comer.

FIG. 4. Growth inhibition of procyclic T. brucei 29-13 cells by interference with histone 2B expression. T. brucei 29-13 cells were transfected with 40 μg of plasmids p2T7 (open circle), p2T7/h2b (closed circle) or p2T7/αtub (open square). Growth rates were determined by measuring cell densities at 0.5, 4, 8, 12, 24, and 32 h after transfection. Cell densities represent the average of two independent transfections.

FIG. 5. Schematic diagram of the second-generation plasmid, p₂T₇^{Ti}, which can integrate into a rRNA spacer region of the genome and be used for inducing the expression of double-stranded RNA interference (dsRNAi) by tetracycline addition. The plasmid p2T₇^{Ti} contains two opposing, tetracycline-inducible T7 promoters (closed horizontal arrowheads) flanking the foreign DNA (for example, the BIP gene of Trypanosoma brucei). The construct is linearized by digestion with NotI and introduced into T. brucei by electroporation where it integrates into the spacer region between the tandem rRNA genes of the genome. The presence of the integrated plasmid is selected for by resistance to phleomycin mediated by the BLE gene product. Unique cloning sites used for insertion of the foreign DNA are shown. T7 transcription terminators (Ω) are positioned outside the T7 promoters to halt transcription from the T7 RNA polymerase. Black box, tetracycline operator; closed arrowhead, T7 promoter; open arrowhead, rRNA promoter; Ω, T7 transcription terminator; gray box, rRNA spacer region.

FIG. 6. BIP mRNA and BIP protein are lost at different rates in procyclic T. brucei cells expressing BIP dsRNA from integrated p2T7^{Ti}/BIP. BIP mRNA and BIP protein levels in the BIP dsRNA cell line (see legend to FIG. 5) were monitored by northern and western blots, respectively. For determination of BIP mRNA levels by northern blots, total RNA was isolated from the cells at the indicated times after inducing dsRNA expression with tetracycline (1 μg/ml). Five μg of total RNA from each time point was subjected to electrophoresis, transferred to nylon, and hybridized to a ³²P-labelled BIP DNA probe. The hybridization signals were determined by instant image analyses. For determination of BIP protein levels by western blots, total cell lysates were prepared at the indicated times after adding tetracycline. Cell lysates (2×10⁵ cell equivalents) were subjected to SDS-polyacrylamide gel electrophoresis followed by immunoblotting with anti-BIP antisera. Protein bands were visualized via enhanced chemiluminescence.

FIG. 7. RNA interference with BIP expression causes multiple defects prior to death of the cells. Procyclic T. brucei 29-13 cells containing an integrated tetracycline-inducible p2T7^{Ti}/BIP dsRNA expression plasmid were established and cloned by serial dilution. BIP dsRNA expression was induced in the cells by adding tetracycline (1 μg/ml) to the growth media. DIC images of live cells grown in the absence (A) or presence (B and C) of tetracycline (65 hours) were obtained as described in FIG. 6. A number of aberrant morphologies are evident in cells expressing BIP dsRNA, including: (a) detached flagella; (b) posterior extension; (c) mini-trypanosome; (d) cytokinesis defects. The cells do not recover from these aberrant morphologies and die, even if tetracycline is removed. Scale bars indicate 10 μm.

### DETAILED DESCRIPTION OF THE INVENTION

Double-stranded RNA interference (dsRNAi) is a recently discovered phenomenon in which the introduction of double-stranded RNA (dsRNA) in cells results in potent and specific silencing of a corresponding gene by causing highly specific degradation of the corresponding endogenous RNA (Bosher J M, Labouesse M. (2000) Nature Cell Biol 2:E31-E36). dsRNA produces a pronounced decrease or elimination of endogenous mRNA transcripts, and dsRNA-mediated interference has a surprising ability to cross cellular boundaries.

dsRNAi was first described in the nematode Caenorabditis elegans (Fire A, et al. (1998) Nature 391:806-11), and has been subsequently observed in Drosophila (Kennerdell J R, Carthew R W (1998) Cell 95:1017-26) (Misquitta L, Paterson B M. (1999) Proc Natl Acad Sci USA 96:1451-6), hydra (Lohmann J U et al. (1999) Dev Biol 214:211-4), planaria (Sanchez Alvarado A, Newmark Pa. (1999) Proc Natl Acad Sci USA 96:5049-54), zebrafish (Wargelius A, et al. (1999) Biochem Biophys Res Commun 263:156-61) (Li Y X, et al. (2000) Dev Biol 217:394-405), mice (Wianny F, Zemicka-Goetz M. (2000) Nature Cell Biol 2:70-75) and trypanosomes (Ngo H, et al. (1998) Proc Natl Acad Sci USA 95:14687-92). In most of these organisms, the presence of dsRNA longer than 500 base pairs specifically suppresses the expression of a gene with a corresponding DNA sequence, but has no effect on genes unrelated in sequence. Because dsRNAi dominantly interferes with gene expression only in a sequence-specific manner at the RNA level, this phenomenon can be exploited to generate knockout phenotypes without creating mutations in the target gene itself. dsRNAi can significantly decrease the time and effort needed to generate knockouts in these and other diploid organisms.

dsRNAi holds particular promise for organisms previously considered not to be amenable to genetic analysis or manipulation, such as the Trypanosomatid protozoan parasites, including Trypanosoma brucei, Trypanosoma cruzi, several Leishmania species, Toxoplasma gondii and several Plasmodium species, all of which are the causative agents of human disease. These protozoan parasites are diploid throughout their life cycle, all except T. gondii lack easily manipulated sexual cycles, and all have many genes represented in multiple copies. All of these factors render traditional gene knockout strategies cumbersome, if not impossible. In addition, it appears that mRNA abundance in Trypanosoma and Leishmania is primarily, if not solely, regulated at the level of RNA metabolism, rather than at the level of transcription initiation as is the case in most eukaryotic organisms.

Ngo and colleagues have shown that introducing double-stranded RNA (dsRNA) derived from the α-tubulin gene (α-tub) into T. brucei cells caused the endogenous α-tub message to be degraded (Ngo H, et al. (1998) Proc Natl Acad Sci USA 95:14687-92). Loss of α-tub RNA caused the cells to round up (a phenotype referred to as FAT) and interfered with cell division (Ngo H, et al. (1998) Proc Natl Acad Sci USA 95:14687-92). Several methods were used successfully to generate dsRNA in T. brucei cells. First, an α-tub DNA fragment was cloned into an expression plasmid as an inverted repeat separated by a segment of unique sequence. Transcription of this plasmid presumably yielded a single transcript containing both sense and antisense RNA, which then annealed to form a hairpin loop containing dsRNA. Second, cells were cotransfected with two expression plasmids containing the α-tub fragment in the sense or antisense orientation. Third, sense and antisense α-tub RNA was made in vitro, annealed, and transfected into T. brucei cells. All three methods produced FAT cells, but the first two methods required at least two cloning steps and the last method required extensive in vitro manipulation prior to transfection. The current techniques to generate dsRNAi require time-consuming in vitro manipulations or multiple cloning steps. None of the methods have the ability to generate dsRNA in a random, sequence-independent manner, a property that would allow dsRNAi to be used, for example, in genetic screens in T. brucei and other protozoan parasites.

The present invention involves a new method to generate double-stranded RNA interference (dsRNAi). Two-promoter vectors are made that can be used to generate knockout phenotypes in a target organism, such as T. brucei, via dsRNAi. The two promoters are arranged as an inverted repeat that allows transcription of both strands of the DNA sequence of interest located between them. The two promoters are oriented towards each other so that both strands of DNA are transcribed, thus producing both sense and antisense RNA that anneal to form dsRNA. Multiple unique restriction sites are located between the promoters, allowing researchers to create plasmid constructs capable of generating dsRNAi in a single step. To generate dsRNAi, the plasmid is introduced into cells where it is transcribed on both strands, forming dsRNA and activating the dsRNAi pathway.

The present system is used to knock out gene expression in organisms that are sensitive to dsRNAi. Random DNA sequences can be inserted into the two-promoter plasmids to create a library of plasmids that will interfere with expression of specific genes. The library of plasmids can be introduced into cells to create random knockout phenotypes. Specific phenotypes can be selected and the gene responsible for generating the phenotype can be recovered by isolating the entire plasmid or by amplifying the DNA insert using the polymerase chain reaction (PCR) and primers specific to the promoter sequence.

The present system has enabled genetic screens to be performed in organisms such as trypanosomes that were not previously amenable to such approaches. Using this approach, for example, knockout phenotypes in T. brucei for α-tub, pfra, gfp, fla1, and histone 2B were quickly generated. The two-promoter system simplifies the production of constructs that generate double-stranded RNA since only a single cloning step is needed. In addition, DNA from cDNA or genomic DNA libraries can be inserted between the two promoters, thus allowing random knockout phenotypes to be generated in the target organism using dsRNAi.

The two-promoter approach for generating dsRNAi is highly applicable to the biopharmaceutical industry. The technique allows a researcher to screen for essential genes and thus to identify potential drug targets in human pathogens such as Trypanosoma, Leishmania, Toxoplasma, and Plasmodia species. The technique can also be used to knock out specific genes in the above organisms and to create attenuated organisms that are used as live vaccines.

This technique overcomes several problems associated with creating gene knockouts in Trypanosoma, Leishmania, Toxoplasma, and Plasmodia and other organisms sensitive to dsRNAi. These organisms are diploid, meaning they have two copies of their genetic material. To knock out specific genes, both copies have to be deleted, which requires extensive cloning, multiple selectable marker genes, and many months of work. In contrast, dsRNAi is a dominant phenomenon that works at the level of RNA expression. A single plasmid construct can be used to generate dsRNAi, which significantly reduces the time needed to obtain knockout phenotypes. The current methods to generate dsRNA require the same DNA sequence to be cloned into an expression plasmid in both the sense and antisense orientation. Because this arrangement is unstable, a region of spacer DNA must be included to separate the sense and antisense portions.

Alternatively, the DNA fragment can be cloned into two separate expression plasmids, one that expresses the sense strand and one that expresses the antisense strand. Both plasmids are then introduced into the same cells to produce dsRNA. A third method is to prepare sense and antisense RNA in vitro, anneal it to form dsRNA, and transfect it into cells. All three require at least two cloning steps, the first approach has problems with plasmid stability, the second approach has difficulty ensuring that each cell receives both plasmids, and the last approach requires extensive in vitro manipulation. In addition, none of these techniques can be used to easily generate dsRNA from a random DNA sequence. The present approach overcomes these limitations by creating a plasmid with two promoters that allow sense and antisense RNA to be made from the same DNA sequence. Plasmids that are used to generate dsRNAi are created in one cloning step. There is no problem with stability because there are no extensive regions of repeated DNA sequence. No in vitro manipulation is needed because the plasmid is transcribed within the cell.

DNA encoding the double-stranded RNA can be prepared by any suitable method, including, for example, restriction and cloning of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Once the target nucleic acid is identified, it can be isolated according to standard methods known to those of skill in the art (see, e.g., Sambrook et al. (1989) Molecular Cloning: A Laboratory Manual, 2nd Ed., Vols. 1-3, Cold Spring Harbor Laboratory; Berger and Kimmel (1987) Methods in Enzymology, Vol. 152: Guide to Molecular Cloning Techniques, San Diego: Academic Press, Inc.; or Ausubel et al. (1987) Current Protocols in Molecular Biology, Greene Publishing and Wiley-Interscience, New York).

Effective Promoters

Promoters, as used herein, are specific DNA sequences at which RNA polymerases bind and begin the synthesis of RNA, i.e., begin transcription. Many different promoters may be used in the present invention. Both promoters in the plasmid may be the same, or may be two different promoters. For example, the ribosomal RNA (rRNA) promoter and the bacteriophage T7 promoter are effective in the two-promoter system in T. brucei. A specific promoter may be selected in order to maximize the desired goal. For example, the rRNA promoter was found to be less effective at generating dsRNAi than the T7 promoter, but can be used in any T. brucei cell line. The T7 promoter generated dsRNAi very efficiently, but was functional only in engineered T. brucei cell lines that express the T7 RNA polymerase. The T. brucei promoters for the variant surface glycoprotein (VSG) gene and the procyclic acidic repetitive protein (PARP) gene may be used as well. Still other eukaryotic promoters can also be used. Other pairs of foreign promoters and their corresponding RNA polymerase function in the same manner as T7 RNA polymerase and the T7 promoter. These RNA polymerase/promoter pairs include the T3 RNA polymerase and the T3 promoter, and the SP6 RNA polymerase and the SP6 promoter. As with the T7 promoter, the T3 and SP6 promoters will only function in T. brucei cell lines engineered to express T3 RNA polymerase or SP6 RNA polymerase, respectively. The promoters could also function in a variety of combinations. For instance, a head-to-head arrangement of T7 and T3 promoters could be used to generate dsRNAi in a cell line expressing the T7 and T3 RNA polymerases.

The experiments involving T. brucei and its rRNA promoters indicate that the rRNA promoters derived from T. cruzi, the Leishmania species, Toxoplasma gondii and the Plasmodium species can be applied in the two-promoter system in these organisms. In addition, the T7, T3, and SP6 promoters are effective in T. cruzi, Leishmania, T. gondii and Plasmodium cell lines engineered to express T7 RNA polymerase, T3 RNA polymerase, or SP6 RNA polymerase, respectively.

Exemplary Plasmids

Many different plasmids may be used in the present invention. For example, a modified pBluescriptII SK(−) plasmid (Stratagene) was engineered to contain two-T7 promoters. Further, a two-rRNA promoter plasmid was derived from the T. brucei expression plasmid pHD496 (Biebinger S, et al. (1996) Nucleic Acids Res. 24:1202-11). Similar plasmids can be used for T. cruzi, Leishmania, T. gondii and Plasmodia.

The plasmid may be constructed so that it is capable of integrating into the genome of the pathogen cells. The advantage of this embodiment is that the plasmid's genetic information would then be permanently maintained in the genome through cell division. One genomic location into which the plasmid can be integrated without interfering with an endogenous genetic function is the ribosomal RNA (rRNA) gene locus. Because eukaryotic genomes contain many tandem rRNA genes, the disruption of a single rRNA gene or intergenic region does not substantively destroy the overall production of the rRNA gene products. Other genomic locations into which the plasmid can be integrated are the genomic sites of other tandem gene families, such as those encoding tubulins, histones, actins, because, again, disruption of one of these genes or intergenic regions does not substantively affect the overall production of the gene family. Still other potential plasmid integration sites are the intergenic regions between genes that are not essential for the function of those genes.

In addition, the plasmid can be constructed in such a way so that synthesis of the double-stranded RNA can be regulated (stimulated or inhibited) by the addition of a compound. For example, a plasmid could be constructed containing tetracycline-inducible promoters as the two opposing promoters. Immediately downstream of each of the two promoters, a tetracycline operator region could be inserted. In the absence of tetracycline the tetracycline repressor protein binds to these operator sites, preventing transcription. When tetracycline is present, it binds to the repressor and the repressor-tetracycline complex is unable to interact with the operator region. This allows transcription from the opposing T7 promoters to extend through both strands of the cloned target DNA to generate the dsRNA.

Other inducible promoters, and their corresponding inducers could be used to practice the present invention. For example, the bacterial lactose operator could be used such that the adjacent promoter would be induced by the addition of isopropylthiogalactoside (IPTG). Another example utilizing a different molecular mechanism is the placement of a transcription termination sequence immediately downstream of the promoter. The termination sequence is flanked by short regions that are recognized by excision- or “flipase” enzymes. Transcription would be terminated until the termination sequence is deleted or inverted via induction of the expression of an excision- or “flipase” enzyme that recognizes the flanking regions and excises or inverts (“flips”) the termination sequence. The Cre-loxP system is one example of this excision/inversion system in which Cre is the enzyme and loxP is the recognition sequence of Cre that would flank the transcription termination sequence.

Strategies for Use of the Two-promoter System

The two-promoter system can be used in two complementary ways. First, because of the ease with which genes can be cloned into the two-promoter vectors, genes of T. brucei, T. cruzi, Leishmania, T. gondii and Plasmodia can be screened in a specific or random manner to identify those genes that are essential for growth of these pathogens in their mammalian hosts. Second, once essential genes have been identified, the two-promoter system can be used to inhibit expression of one or more of those genes and thus prevent the parasite from growing in the mammalian host. For, example, cells of bloodstream form T. brucei, T. cruzi and Leishmania all express a zinc metalloprotease called GP63 on their cell surface. This protease is thought to help protect the parasites from complement-mediated lysis. Without the protease, the parasites will be killed in the mammalian bloodstream. Because GP63 is unlikely to be necessary for the parasites to grow in cell culture, engineered cell lines can be established that use the two-promoter system to inhibit GP63 expression. The engineered Trypanosoma or Leishmania cells that lack GP63 can then be injected into humans or cattle. These genetically attenuated cells will not grow, but will stimulate an immune response that will serve to prevent subsequent infections.

The expression vectors (i.e., expression plasmids) can be transferred into the chosen protozoan parasite cells by the well-known method of electroporation, or by other known methods. Cells transformed by the expression vectors can be selected by growth in culture media containing an antibiotic whose corresponding antibiotic resistance gene is encoded on the expression vector. These antibiotic resistance genes include, but are not limited to, genes encoding neomycin phosphotransferase, which provides resistance to G418; hygromycin phosphotransferase, which provides resistance to hygromycin; bleomycin resistance protein, which provides resistance to phleomycin; and puromycin acetyltransferase, which provides resistance to puromycin.

Vaccine Preparation and Administration

To prepare a vaccine, the protozoan parasite cells bearing the expression vector are isolated. The amount of these cells may then be adjusted to an appropriate concentration, optionally combined with a suitable vaccine adjuvant, and packaged for use. Suitable adjuvants include but are not limited to surfactants, e.g., hexadecylamine, octadecylamine, lysolecithin, dimethyldioctadecylammonium bromide, N,N-dioctadecyl-N′-N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, MPL, aimethylglycine, tuftsin, oil emulsions, alum, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993).

To immunize a subject, the protozoan cell containing the expression vector is administered parenterally, usually by intramuscular or subcutaneous injection in an appropriate vehicle. Other modes of administration, however, such as oral delivery or intranasal delivery, are also acceptable. Vaccine formulations will contain an effective amount of the active ingredient in a vehicle. The effective amount is sufficient to prevent, ameliorate or reduce the incidence of a protist colonization in the target mammal. The effective amount is readily determined by one skilled in the art. The active ingredient may typically range from about 1% to about 95% (w/w) of the composition, or even higher or lower if appropriate. The quantity to be administered depends upon factors such as the age, weight and physical condition of the animal or the human subject considered for vaccination. The quantity also depends upon the capacity of the animal's immune system to synthesize antibodies, and the degree of protection desired. Effective dosages can be readily established by one of ordinary skill in the art through routine trials establishing dose response curves. The subject is immunized by administration of the attenuated vaccine in one or more doses. Multiple doses may be administered as is required to maintain a state of immunity to the protist.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

The following examples are intended to illustrate but not limit the invention.

### EXAMPLE 1

### Two-promoter System in T. brucei

Plasmid Construction

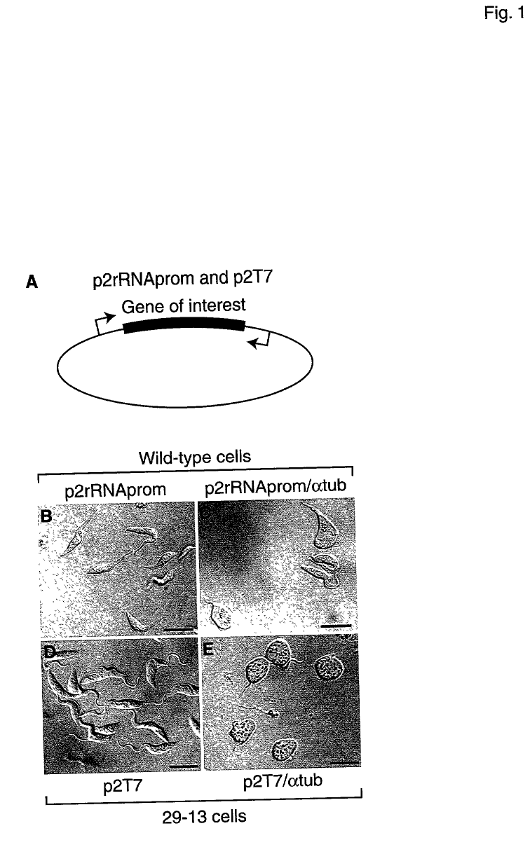

To generate p2rRNAprom (FIG. 1A), a 292-bp fragment containing the T. brucei rRNA promoter was PCR-amplified with primers that added XhoI and BamHI sites to the ends and inserted into the SalI and BamHI sites of pHD496 in the opposite orientation to the rRNA promoter already present (Biebinger S, et al. (1996) Nucleic Acids Res 24:1202-11). Plasmid p2rRNAprom/αtub was created by inserting a 486-bp PCR fragment of T. brucei α-tub (60 bp of the 5′ UTR and 426 bp of coding region) into the HindIII and BamHI sites of p2rRNAprom. A second T7 promoter in the opposite orientation to the T7 promoter already present was added to pBluescriptII SK(−) by annealing oligos 5′-CGTAATACGACTCACTATAGGGCAGCT-3′ and 5′-GCCCCTATAGTGAGTCGTATTACGAGCT-3′ and ligating into the SacI site of pBluescriptII SK(−) to give p2T7 (FIG. 1A).

The 486-bp α-tub fragment described above was PCR-amplified with primers that added HindIII and XbaI sites to the ends and ligated into the HindIII and XbaI site of pBluescriptII SK(−) and p2T7, yielding pBS/αtub and p2T7/αtub, respectively. The α-tub fragment was excised from p2T7/αtub with BamHI and HindIII, blunted with T4 DNA polymerase, and ligated into the EcoRV site of pBluescriptII SK(−) to generate pBS/αtub-AS. Plasmid p2T7/pfra was made by PCR-amplifying a 453-bp fragment from the paraflagellar rod protein A gene (nucleotides 546 to 1099) with primers that added EcoRI and XbaI sites to the ends and ligating into the EcoRI and XbaI sites of p2T7. A 758-bp fragment from plasmid pHD:HX-GFP (Hill K L, et al. (1999) J Cell Sci 112:3091-3101) containing the entire GFP coding sequence was inserted into the BamHI and HindIII sites of p2T7, yielding plasmid p2T7/gfp. A 750-bp XbaI/BamHI fragment from pHD:HX-GFP was inserted into the XbaI and BamHI sites of pBluescript to produce pBS/gfp-AS.

To create pLEW82:GFP, a HindIII/BamHI fragment from pHDHXGFP that included the GFP coding sequence was substituted for the luciferase gene in pLEW82 (Wirtz E, et al. (1998) Nucleic Acids Res 26:4626-34). Plasmids p2T7/fla1 and pBS/fla1-AS were created by PCR-amplifying a 366-bp fragment (nucleotides 318 to 684 from the cDNA) of the T. brucei fla1 gene from genomic DNA with primers that added XbaI and HindIII sites to the ends and ligating into the corresponding sites of p2T7 and pBluescriptII SK(−), respectively. Plasmid p2T7/h2b was created by PCR-amplifying a 539 bp fragment (nucleotides 30 to 569) of the T. brucei histone 2B gene with primers that added EcoRI and XbaI sites to the ends and ligating into the corresponding sites in p2T7.

Cell Lines

The procyclic T. brucei cell lines 29-13 (a gift from Dr. G. A. M. Cross, Rockefeller University (Wirtz E, et al. (1999) Mol Biochem Parasitol 99:89-101)) and YTAT 1.1 (a gift from Dr. E. Ullu, Yale University) were propagated in Cunningham's S M media supplemented with 10% heat inactivated fetal calf serum. The inducible GFP-expressing cell line 82GFP was obtained by transfecting 29-13 cells with linear pLEW82:GFP and selecting for resistance to 2.5 mg ml⁻¹ phleomycin.

Transfections and Microscopy

DNA transfections (40 or 60 μg) were performed as described (Hill K L, et al. (1999) J Cell Sci 112:3091-3101). Transfected cells were immediately transferred to fresh media. For transfections of 82GFP cells, 1 μg ml⁻¹ tetracycline was included in the media to induce GFP expression. Fluorescent and differential interference contrast (DIC) images were obtained with a Zeiss LSM 510 Laser Scanning Confocal Microscope using 63× or 100× oil immersion objectives. DIC and fluorescent images were captured simultaneously on separate channels using Zeiss LSM Image Browser Software and merged in Adobe PhotoShop. Images were adjusted for brightness and contrast and cropped using Adobe PhotoShop (Adobe Systems, Inc., San Jose, Calif.). Growth curves were generated by measuring cell density with a hemocytometer at 0.5, 4, 8, 12, 24, and 32 h after transfection. Two independent transfections were performed for each plasmid.

Flow Cytometry

82GFP cells were transfected with 40 μg of plasmid p2T7, p2T7/gfp, or pBS/gfp-AS as described above and transferred to fresh media containing 1 μg ml⁻¹ tetracycline. At 24 h after transfection, 82GFP cells were collected by centrifugation at 1500×g, washed with PBS (70 mM NaCl, 47 mM Na₂HPO₄, 3 mM NaH₂PO₄, pH 8.0), and suspended in PBS at 1×10⁶ cells ml⁻¹. Propidium iodide (2 μg ml⁻¹ final concentration) was added to distinguish live and dead cells. Flow cytometry was performed on a Becton Dickinson FACScan. GFP fluorescence was excited at 488 nm and monitored using an FITC filter.

Results

Inhibition of α-tubulin Gene Expression Using Two Head-to-head Promoters

To simplify the generation of dsRNA in T. brucei, the potential for a plasmid bearing two T. brucei ribosomal RNA (rRNA) promoters arranged head-to-head (p2rRNAprom, FIG. 1A) to cause dsRNAi was examined. This arrangement was predicted to produce sense and antisense RNA from the sequence located between the promoters, which could then form dsRNA. The ability of this plasmid to cause dsRNAi was tested by inserting a 486-bp fragment from α-tub between the two promoters (p2rRNAprom/αtub). Wild-type procyclic (insect stage) T. brucei cells were transfected with either the parental plasmid p2rRNAprom or p2rRNAprom/αtub and monitored for the appearance of FAT cells. At 12 h after transfection, all cells transfected with p2rRNAprom appeared wild-type (FIG. 1B and Table 1), whereas approximately 16% of the p2rRNAprom/αtub-transfected cells showed the FAT cell phenotype (FIG. 1C and Table 1). Thus, a head-to-head arrangement of two rRNA promoters can be used to generate dsRNAi in T. brucei.

TABLE 1
Percentage of FAT cells 12 h after transfection.

| Plasmid | FAT |
|---|---|
| p2rRNAprom | 0% |
| p2rRNAprom/αtub | 16% |
| p2T7 | 0.3% |
| pBS/αtub | 1.4% |
| p2T7/αtub | 85% |
| pBS/αtub-AS | 46% |
| linear p2T7 | 1% |
| linear p2T7/αtub | 70% |
| linear pBS/αtub-AS | 40% |

Plasmids p2rRNAprom and p2rRNAprom/αtub (60 μg) were transfected into 2.5×10⁷ wild-type YTAT 1.1 cells by electroporation. The remaining plasmids (40 μg) were transfected into cell line 29-13 (2.5×10⁷ cells), which expresses the T7 RNA polymerase. pBS/αtub and pBS/αtub-AS express sense and antisense α-tub RNA, respectively, from the single T7 promoter present in pBluescriptII SK(−). p2T7, p2T7/αtub, and pBS/αtub-AS were linearized by digestion with NgoMIV, phenol/chloroform extracted, and ethanol precipitated prior to transfection of 29-13 cells.

To enhance the production of dsRNAi, a stronger promoter was selected in anticipation that greater expression would increase the amount of dsRNA. Because of the lack of RNA polymerase II promoters in T. brucei, the bacteriophage T7 promoter and a previously developed T. brucei cell line engineered to express the T7 RNA polymerase (cell line 29-13) (Wirtz E, et al. (1999) Mol Biochem Parasitol 99:89-101) were used. A modified pBluescript plasmid with two head-to-head T7 promoters (p2T7) was created and tested for its ability to generate dsRNAi. Procyclic 29-13 cells were transfected with a derivative of p2T7 that contained the α-tub fragment (p2T7/αtub) and monitored for the appearance of FAT cells. At 12 h after transfection, 85% of the p2T7/αtub-transfected cells were FAT (FIG. 1E and Table 1). p2T7/αtub induced the appearance of FAT cells only when the target cells expressed T7 RNA polymerase, indicating that transcription of the plasmid was required for the effect. Neither p2T7 nor pBS/αtub, in which a single T7 promoter drives expression of sense α-tub RNA, produced FAT cells when introduced into 29-13 cells, demonstrating that the antisense strand must be transcribed (FIG. 1D and Table 1). Surprisingly, pBS/αtub-AS, which has a single T7 promoter driving expression of antisense α-tub, also generated FAT cells, though less effectively than p2T7/αtub (46% vs. 85%, see Table 1). Generation of FAT cells did not depend on the presence of circular plasmid DNA, which could potentially yield very long transcripts of unknown structure. Linear p2T7/αtub and pBS/αtub-AS were as effective as their circular counterparts at generating FAT cells (Table 1). Thus, by using the strong T7 promoter and a cell line that expressed T7 RNA polymerase, knockouts in T. brucei were efficiently generated.

Interference with Paraflagellar Rod Protein a Expression Using Plasmid p2T7/pfra

The paraflagellar rod A (pfra) gene was targeted next to determine if this technology could be used to interfere with expression of other T. brucei genes. The PFRA protein is a major constituent of a rod-like structure that extends the length of the flagella and has been shown to be required for T. brucei motility (Bastin P, et al. (1998) Nature 391:548). Cells lacking PFRA are paralyzed and sink to the bottom of the culture dish (Bastin P, et al. (1998) Nature 391:548). To determine if this phenotype could be generated via the two-T7 promoter approach, a 453-bp fragment of the pfra gene was cloned into p2T7 and introduced into 29-13 cells. At 24 h after transfection, a large portion of the cells settled to the bottom of the culture dish. When examined under the light microscope on a hemocytometer (to allow movement), many of the cells transfected with p2T7/pfra were stationary and beat their flagella more slowly than cells transfected with p2T7, consistent with loss of PFRA. Thus, by using a head-to-head arrangement of two promoters to produce dsRNA, the phenotypes of bothpfra and α-tub knockouts were recreated.

Inhibition of GFP Accumulation with Plasmid p2T7/gfp

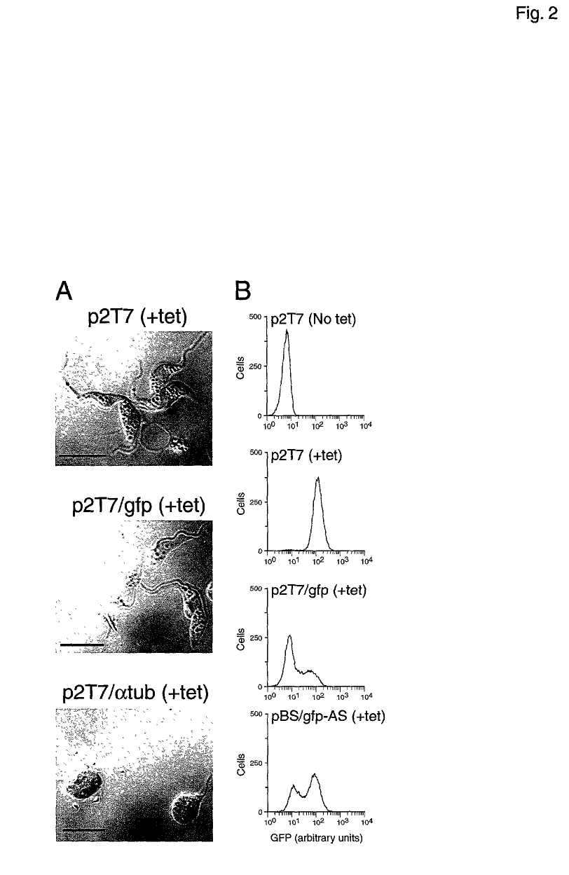

To further evaluate the utility of the two-promoter approach, an attempt was made to interfere with expression of the green fluorescent protein (GFP) in cells engineered to express both GFP and T7 RNA polymerase (cell line 82GFP). 82GFP cells were transfected with p2T7, p2T7/gfp, or p2T7/αtub, incubated with tetracycline to induce gfp expression, and monitored for green fluorescence and the FAT cell phenotype by confocal microscopy. Transfection with p2T7 did not affect gfp expression and the cells became bright green (FIG. 2A). In contrast, p2T7/gfp dramatically reduced the number of green fluorescent cells, demonstrating that GFP accumulation was inhibited (FIG. 2A). The reduction in GFP varied from a modest decrease to complete inhibition among cells in the same transfection. Interference with gfp expression was specific to p2T7/gfp. p2T7/Itub caused 80% of the cells to become FAT, but did not affect green fluorescence (FIG. 2A).

To better quantify the levels of GFP, transfected 82GFP cells were analyzed by flow cytometry. In the absence of tetracycline, 82GFP cells did not express GFP and had only background levels of green fluorescence (FIG. 2B). Addition of tetracycline induced GFP expression and caused 97% of the cells to become bright green (FIG. 2B). p2T7/gfp reduced both the number of green fluorescent cells and the intensity of the fluorescence (FIG. 2B). Only 24% of the p2T7/gfp-transfected cells displayed green fluorescence of equal intensity to the p2T7-transfected cells, whereas 76% of the p2T7/gfp-transfected cells showed reduced or no fluorescence. Some cell-to-cell variability in the amount of reduction in GFP fluorescence was apparent, possibly due to the number of p2T7/gfp plasmids per cell or to the amount of dsRNA produced. As observed with α-tub, antisense gfp also interfered with GFP expression. However, compared to p2T7/gfp, pBS/gfp-AS, which expresses antisense gfp from a single T7 promoter, inhibited GFP accumulation in fewer cells and to a lesser extent (FIG. 2B). Fifty-seven percent (57%) of pBS/gfp-AS-transfected cells had fluorescent intensities equal to those of p2T7-transfected cells, while 43% had reduced GFP fluorescence.

Interference with fla1 Expression Using Plasmid p2T7/fla1

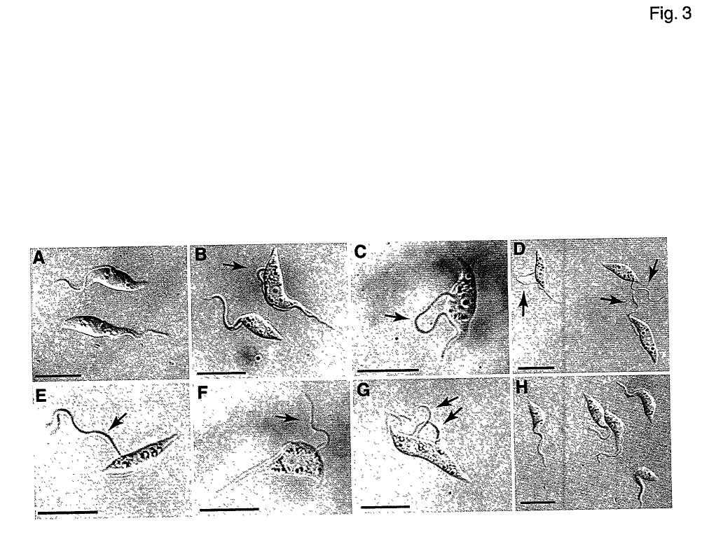

The ability of the p2T7 vector to generate knockout phenotypes for genes not previously characterized in T. brucei was next tested. It was first attempted to inhibit expression of the T. brucei flagellum-adhesion glycoprotein 1 (fla1) gene (Nozaki T, et al. (1996) Mol Biochem Parasitol 82:245-55). FLA1 is a T. brucei homolog of a 72-kDa glycoprotein localized to the junction between the flagellum and the cell body of Trypanosoma cruzi, the related trypanosomatid parasite that causes Chagas disease in Latin America (Cooper R, et al. (1993) J Cell Biol 122:149-56) (Haynes P A, et al. (1996) J Cell Sci 109:2979-88). Previously, when one of the two copies of T. brucei fla1 was deleted, no phenotype was observed (Nozaki T, et al. (1996) Mol Biochem Parasitol 82:245-55). However, attempts to delete both copies of fla1 were unsuccessful, suggesting that fla1 is essential in T. brucei (Nozaki T, et al. (1996) Mol Biochem Parasitol 82:245-55). To determine if fla1 knockout phenotypes via dsRNAi could be obtained, 29-13 cells were transfected with p2T7 or p2T7/fla1. About 50% of the cells transfected with p2T7/fla1 exhibited detached flagella (FIGS. 3B-G), demonstrating that FLA1 is required for flagellar attachment. As noted above, cells transfected with p2T7 appeared normal (FIG. 3A). In contrast to antisense α-tub and gfp, expression of antisense fla1 did not produce mutant phenotypes (FIG. 3H).

Four types of cells with detached flagella were observed. Cells with single partially or fully detached flagellum were seen most frequently. The partially detached flagellum formed a loop, which typically began near the flagellar pocket, the site where the flagellum emerges from the cell body (FIGS. 3B, C). The size of the loop varied with the amount of detached flagellum. Over time the average size of the detached loops appeared to increase as more of the flagellum separated from the cell body (FIGS. 3C, D). In a smaller number of cells the flagellum completely separated from the cell body except for the point of attachment at the flagellar pocket near the posterior end of the cell (FIGS. 3D, E). The completely detached flagellum continued to beat rapidly, but cell motility was impaired. Although in most cases flagella began to detach from the posterior end, in at least some cases the flagellum detached from the anterior end first (not shown), indicating there is no absolute order by which the flagellum separates from the cell body.

Although less common than the first two types, cells with one detached flagellum and one attached flagellum were also observed (FIG. 3F). Since the detached flagellum was always posterior to the attached flagellum and varied in size, the detached flagellum was most likely the newly synthesized one (Sherwin T, et al. (1989) Philosophical Transactions of the Royal Society of London—Series B: Biological Sciences 323:573-88). Thus, flagellar synthesis in T. brucei does not require flagellar attachment to the cell body. Furthermore, the newly synthesized flagellum is more sensitive to loss of FLA1 protein than the existing one, suggesting that these cells possess sufficient FLA1 protein to maintain attachment of the first flagellum, but not enough to create an attachment for the second. An even smaller number of cells displayed two completely detached flagella (FIG. 3G). The fate of these cells is unclear. Although in some cases these cells appeared to be nearing completion of cytokinesis, many examples of cells that appeared unable to divide properly were observed. Because they were unable to generate a double fla1 knockout in T. brucei, Nozaki et al. originally proposed that fla1 is essential in T. brucei and may be required for kinetoplast separation and cell cleavage (Nozaki T, et al. (1996) Mol Biochem Parasitol 82:245-55). The present observations are consistent with this proposal.

Interference with Histone 2B Expression Using p2T7/h2b

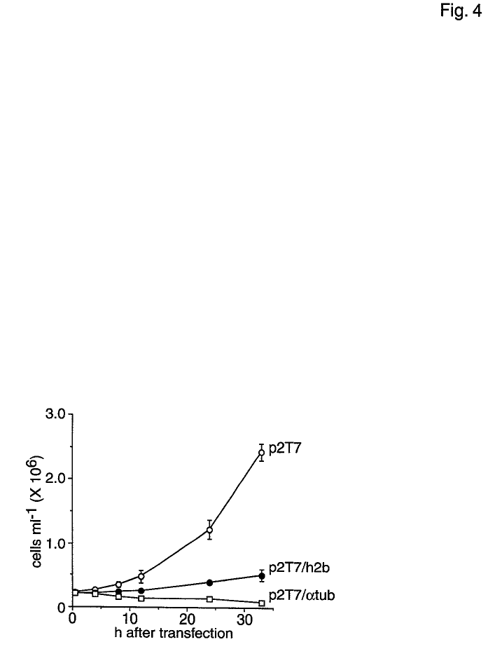

The two-T7 promoter system was also used to interfere with histone 2B (h2b) expression. The protein product of h2b, H2B, is a small, highly conserved, positively charged protein in eukaryotic cells that binds tightly to double-stranded DNA. Based on its essential role in yeast (Rykowski M C, et al. (1981) Cell 25:477-87) and its importance in chromatin structure, h2b is also likely to be required for growth in T. brucei. To determine if loss of H2B affected T. brucei growth, 29-13 cells were transfected with a derivative of p2T7 that contains 539 bp of T. brucei h2b (p2T7/h2b). Cells transfected with p2T7/h2b were motile and appeared wild-type, but grew slowly compared to cells transfected with p2T7 (FIG. 4). A slight increase in cell number was observed in p2T7-transfected cells at 32 h after transfection. It was not clear if this increase resulted from division of untransfected cells or slow growth of cells harboring the p2T7/h2b plasmid. Transfection with p2T7/αtub also caused a severe reduction in growth due to defects in cytokinesis resulting from loss of α-tub (Ngo H, et al. (1998) Proc Natl Acad Sci USA 95:14687-92). Based on this experiment, h2B appears to be required for growth in T. brucei, which confirms that novel knockout phenotypes can be obtained in T. brucei using the two-promoter approach.

Discussion

The present inventors have shown that head-to-head promoters can be used to create knockout phenotypes in T. brucei. The effect appears to be mediated by dsRNAi since transcription of both strands of the gene of interest was required to most efficiently generate knockout phenotypes. Using the two-T7 promoter vector, knockout phenotypes for α-tub, gfp, pfra, fla1, and histone 2B were quickly generated. Also, roles forflal in flagellar attachment and for histone 2B in cell growth in T. brucei were demonstrated. The present experiments with these genes illustrate the primary advantages of the two-promoter approach. Plasmid constructs that generate dsRNA can be created with a single, simple cloning step, which significantly reduces the amount of time and effort needed to obtain knockout phenotypes in T. brucei. Because highly efficient transfection methods are available for procyclic T. brucei cells, essential genes can be knocked out in a transient assay. Furthermore, random DNA sequences from cDNA or genomic libraries can be inserted between the two promoters, which allows dsRNAi to be used to create random knockout phenotypes. Thus, the two-promoter approach is the first convenient random knockout technology in T. brucei and forms the basis for genetic selection schemes that have not been practical to date. This approach is useful for related parasites such as Trypanosoma cruzi and Leishmania and other organisms that are sensitive to dsRNAi.

Both antisense and double-stranded RNA have been reported to silence gene expression in kinetoplastids. Antisense RNA has been used to inhibit expression of the gp63 and A2 genes in Leishmania (Chen D Q et al. (2000) Infect Immun 68:80-6) (Zhang W W, Matlashewski G. (1997) Proc Natl Acad Sci USA 94:8807-11) and the pfra gene in T. brucei, although inhibition of pfra appeared to require integration of the antisense construct at the pfra locus (Bastin P, et al. (1998) Nature 391:548). In all three cases, antisense RNA expression reduced the level of the corresponding endogenous mRNA. In contrast to these reports, Ngo et al. found that double-stranded α-tub RNA, but not antisense α-tub RNA, inhibited α-tub gene expression in T. brucei. The net result was the same, however, as both antisense RNA and dsRNA reduced the level of endogenous mRNA. The present results show that dsRNA is more effective than antisense RNA at inhibiting gene expression in T. brucei. Antisense RNA was approximately half as efficient as dsRNA at generating knockout phenotypes for α-tub and gfp, and did not generate fla1 knockout phenotypes.

### EXAMPLE 2

### Induction of Expression of the Two-promoter System (from p2T7^{Ti}/BIP) Integrated into the Genome of Trypanosoma brucei

Plasmid Construction

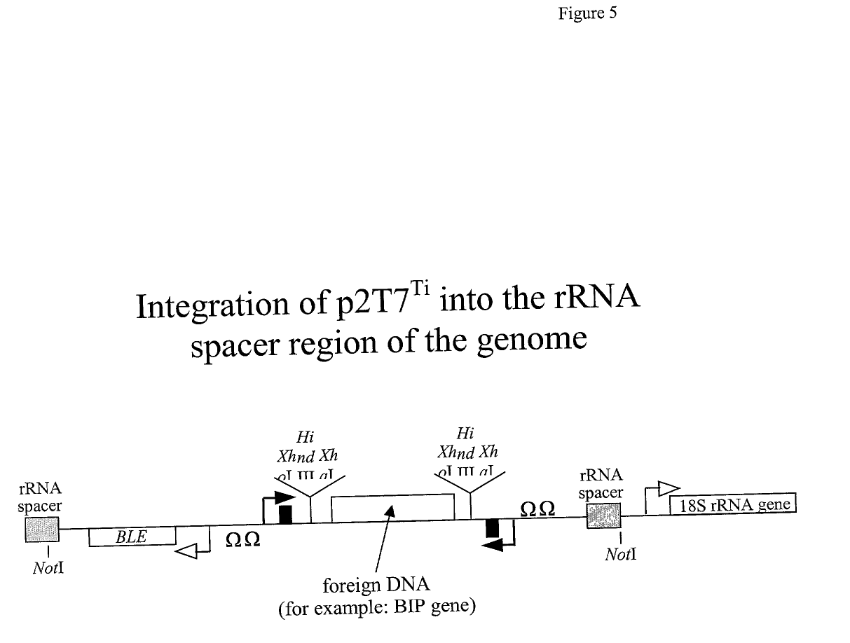

Plasmid p2T7^{Ti}/BIP (FIG. 5) was generated from plasmid pLEW82 (Wirtz E, et al. (1999) Mol Biochem Parasitol 99:89-101) as follows. A SacII/HindIII fragment from pLEW82 was blunted with T4 DNA polymerase and inserted into SalI digested, blunt-ended pLEW82 (plasmid A). The T7 terminators from pLEW82 were subcloned into the PstI site of pBluescriptII SK−. The resulting plasmid was digested with EcoRV and SmaI and the fragment containing the T7 terminators was ligated into the blunted SacII site from plasmid A (plasmid B). The coding sequence of the T. brucei BIP gene was PCR-amplified with primers bearing HindIII and BamHI sites at their 5′ ends and ligated into the corresponding sites of pBluescriptII SK−, giving pBS/BIP-H/B. The BIP gene and the multiple cloning sequence from pBS/GFP-H/B were excised with KpnI and SacI, blunted with T4 DNA polymerase, and ligated into SmaI digested plasmid B (plasmid C). To fuse the T. brucei rRNA promoter to the BLE selectable marker gene, a KpnI/SmaI fragment containing the rRNA promoter from pHD496 and a SmaI/NcoI fragment from pLEW82 containing the actin 5′ UTR and the 5′ end of BLE was inserted into KpnI/NcoI digested pBS/BIP-H/B via triple ligation (plasmid D). The 3′ end of BLE and the 3′ UTR were excised from pLEW82 with NcoI and PstI and cloned into the corresponding sites of plasmid D to regenerate a complete BLE gene (plasmid E). The rRNA promoter-BLE construct from plasmid E was liberated with PstI and KpnI, blunted with T4 DNA polymerase, and ligated into the blunt-ended NheI site from plasmid C, yielding p2T7^{Ti}/BIP.

Cell Lines and Transfections:

Procyclic T. brucei 29-13 cells (T7RNAP NEO TETR HYG) and the bloodstream T. brucei single marker cell line (T7RNAP TETR NEO) were used for tranfection with the plasmid (Wirtz E, et al. (1999) Mol Biochem Parasitol 99:89-101). 29-13 cells were maintained in Cunningham's S M media supplemented with 10% fetal calf serum and were transfected with NotI linearized plasmids (5-10 μg). Log phase cells (5×10⁶ ml⁻¹) were collected by centrifugation, washed with EM (a 3:1 mixture of cytomix and PS [277 mM sucrose, 1 mM MgCl₂ 7 mM KᵢPO₄, pH 7.4]), and suspended in EM at a concentration of 2.5×10⁷ ml⁻¹. 0.45 ml of cells were mixed with 0.1 ml of linearized DNA in a 0.4 cm electroporation cuvette and subjected to two pulses from a Biorad Gene Pulser electroporator set at 1500 V and 25 μfarad. After electroporation, cells (0.2-0.3 ml) were transferred to 4 ml fresh SM+10% FCS and allowed to recover overnight. Stable transformants were selected in 15 μg ml⁻¹ G418, 50 μg ml⁻¹ hygromycin, and 2.5 μg ml⁻¹ phleomycin. Once drug resistant pooled lines were established, clonal lines were obtained by limiting dilution.

The bloodstream single marker line was grown in HMI-9 media (Hirumi, H., and Hirumi, K. (1989) J. Parasitol. 75:985-9) supplemented with 10% FCS and transfected essentially as described for 29-13 cells. Log phase single marker cells (˜1.0×10⁶ ml⁻¹) were collected by centrifugation, washed with EM, and resuspended in EM at a concentration of 2×10⁷ ml⁻¹. 0.45 ml of cells were mixed with 5-10 μg of linerized DNA in a 0.4 cm electroporation cuvette and subjected to a single pulse from a Biorad Gene Pulser electroporator set at 1500 V and 25 μfarad. Cells were transferred to 12 ml of HMI-9+10% FCS and distributed among wells in a 24-well tissue culture plate. After recovering overnight, an equal volume of HMI-9+10% FCS plus 5 μg ml⁻¹ G418 and 5 μg ml⁻¹ phleomycin was added to the wells. Phleomycin-resistant cells typically grew out within 7 days.

Results and Discussion

The plasmid containing two opposing promoters depicted in FIG. 1 can be used to generate double-stranded RNA interference (RNAi) in procyclic T. brucei (LaCount D J, et al. (2000) Mol Biochem Parasitol 111: 67-76). However, two limitations of this first-generation version of the two-promoter plasmid are that (i) it is not maintained episomally (i.e., as a freely replicating plasmid) in T. brucei cells, and (ii) expression of the double-stranded RNA cannot be regulated. Thus, the length of time that dsRNA is expressed from the two-promoter plasmid is dependent on the lifetime of the transiently transfected DNA in the cell, and the dsRNA is only synthesized during this plasmid lifetime and in an unregulated manner.

To overcome these limitations (i.e., to extend the length of time that RNAi can be maintained and to regulate the expression of RNAi), an integratable version of the two-T7 promoter vector, called p₂T₇^{Ti}, was developed that utilizes two tetracycline-inducible T7 promoters to generate sense and anti-sense RNA from the DNA sequences placed between them (FIG. 5). Plasmid p2T7^{Ti} contains two opposing bacteriophage T7 promoters that flank three unique cloning sites in the plasmid (XhoI, HindIII and XbaI). Between each T7 promoter and the cloning sites is a bacterial tetracycline operator region. In the absence of tetracycline the tetracycline repressor protein binds to these operator sites, preventing transcription. However, when tetracycline is present, it binds to the repressor and the repressor-tetracycline complex is unable to interact with the operator region; thus, transcription from the opposing T7 promoters extends through both strands of the cloned foreign DNA to generate the dsRNA. The T7 transcription termination sequences are placed in both strands of the DNA to prevent transcription into the flanking regions, which might inadvertently interfere with the expression of a downstream gene after integration of the plasmid into the genome. The plasmid also contains a T. brucei rRNA gene spacer region with an interior NotI site. The NotI cleavage site is a specific sequence of 8 base pairs, so it occurs rarely in DNA sequences. When the plasmid is cleaved with NotI, both ends of the linearized plasmid contain the rRNA gene spacer sequence, which facilitates its integration into the T. brucei genome following electroporation. Finally, the plasmid contains the bleomycin (phleomycin) resistance gene preceded by the T. brucei rRNA promoter, so that T. brucei cells into whose genome the linearized plasmid has integrated can be selected for by growth in phleomycin.

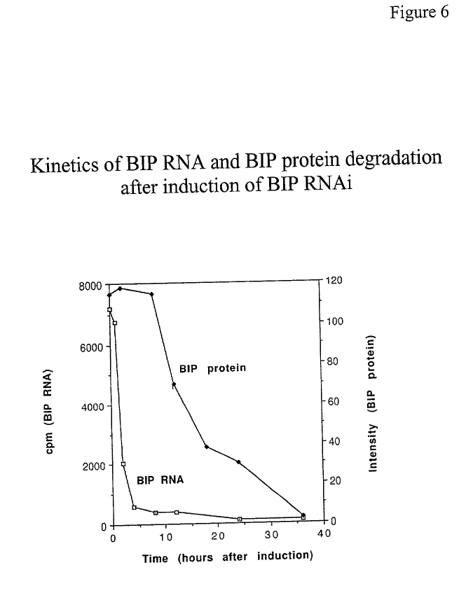

To test the effectiveness and utility of p2T₇^{Ti}, the coding sequence of the T. brucei BIP gene was PCR-amplified and cloned into the multiple cloning site between the two T7 promoters of p2T₇^{Ti} and the resulting plasmid, p2T7^{Ti}/BIP, was linearized and electroporated into T. brucei. BIP is a chaperone protein localized to the endoplasmic reticulum of all eukaryotic cells, including T. brucei (Bangs J D, et al. (1993) J Cell Sci 105:1101-1113). Because BIP is essential in yeast (Normington K, et al. (1989) Cell 57:1223-1236) (Rose M D, et al. (1989) Cell 57: 1211-1221), it was expected that BIP would also be required in T. brucei. It was surprising, therefore, that inhibiting BIP expression via RNAi in transient assays neither caused cell death nor induced a noticeable phenotype. To examine whether BIP truly is dispensable for growth of procyclic T. brucei, stable cell lines that express BIP dsRNA in response to tetracycline were prepared and the rate at which BIP RNA and BIP protein were lost was compared by northern and western blot, respectively (FIG. 6) (LaCount D J and Donelson J E (2001) The Protist (in press)). Within one hour after adding tetracycline, BIP RNA levels began to decrease. By 4 h, BIP RNA was reduced to very low levels and at 48 h, was undetectable. However, BIP protein was easily detected until 48 h after adding tetracycline. Quantitation of the levels of BIP RNA and BIP protein indicated that the half-life of BIP RNA is 1-2 h, whereas that of BIP protein is about 16 h.

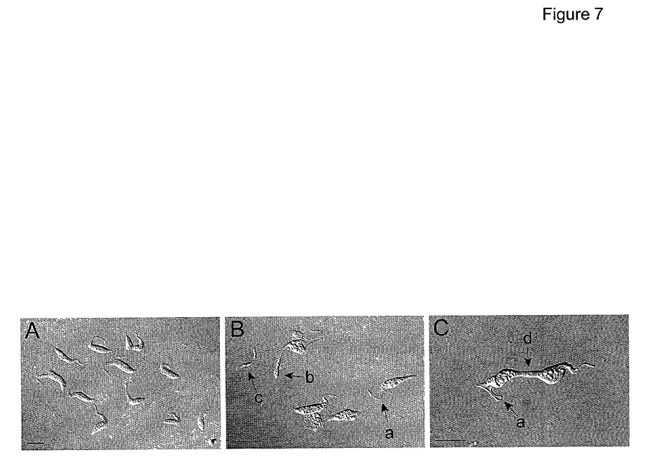

For the first 48 h after tetracycline addition (˜6-8 cell doublings), no obvious change in the morphology or growth of the cells occurred. However, beginning about 48 h after inducing BIP dsRNA expression, a variety of mutant phenotypes were observed (FIG. 7). These phenotypes included cells with partially detached flagella, cells with elongated posterior extensions, mini-trypanosomes that appear to lack nuclei, and cells with defects in cytokinesis. These pleiotropic effects are consistent BIP's role as an ER chaperone protein. In the absence of BIP, many proteins that traffic through the ER would be expected to be improperly folded, which accounts for the varied cellular morphologies observed. The appearance of mutant phenotypes in the cell lines induced to express BIP dsRNA is greatly delayed because BIP protein is degraded much more slowly than BIP RNA. Once the mutant phenotypes develop, however, the cells cannot recover, even if tetracycline is removed, and cell death results.

These results demonstrate that BIP is essential in T. brucei, and illustrates one of the major advantages of using an inducible and integrated two-promoter plasmid, such as p₂T₇^{Ti}, to generate the RNAi that inhibits gene expression. The cells can be allowed to grow for a desired length of time and then RNAi induced by the addition of tetracycline at the appropriate time. Furthermore, the cells can be forced to continue to synthesize the dsRNA through many cell divisions until the essential protein is sufficiently depleted that the cells can no long divide and undergo death.

### EXAMPLE 3

### Targeted dsRNAi in T. brucei, T. cruzi and P. falciparum

Trypanosoma brucei is a single celled protozoan parasite. Its life cycle alternates between growth in an insect vector (the tsetse fly) and a mammalian host. T. brucei lives extracellularly throughout its lifecycle. In the insect, it grows in the midgut, then migrates to the mouthparts. T. brucei is injected into the mammalian bloodstream through the bite of an infected tsetse fly. The parasite replicates in the bloodstream and can be transmitted back to the tsetse fly when a fly bites an infected mammal. T. brucei causes a fatal disease called African trypanosomiasis (sleeping sickness) in humans and a related disease (ngana) in cattle. A head-to-head two-promoter plasmid is generated containing a gene that is active only when the parasite is in the mammalian host, i.e., not when in the insect vector. The plasmid is introduced into the T. brucei. The plasmid does not have a deleterious effect when the parasite is living in the insect or in culture medium, but once the parasite is transmitted to the bloodstream of a mammalian host, the presence of dsRNA prevents expression of the gene that is active only in the mammalian host, thereby killing the parasite. One such gene encodes a protease called GP63 (E1-Fayed, N. and Donelson, J. (1997) J Biol. Chem. 272:26742-48). This engineered parasite is useful as an attenuated, live vaccine.

Trypanosoma cruzi is another single celled protozoan parasite whose life cycle also alternates between growth in an insect vector (the reduviid bug) and a mammalian host. In contrast to T. brucei, however, T. cruzi is an intracellular parasite in its mammalian host and replicates inside a number of different cells of the mammalian host including muscle cells. Thus, T. cruzi contains a number of genes that are expressed predominately in the intracellular stage, i.e., the amastigote stage, that are not expressed, or are expressed at only a very low level, in the insect stage or in culture medium. A head-to-head two-promoter plasmid is generated containing a gene that is active only in the intracellular amastigote stage of the parasite. One such gene encodes a surface protein of the amastigote stage called amastin (Teixeira, S. et al. (1994) J. Biol. Chem. 269:20509-16). Thus, the dsRNA encoded on the two-promoter plasmid is directed against mRNA of a gene that is active only in the amastigote stage, thereby killing the intracellular parasite. This engineered parasite is useful as an attenuated, live vaccine.

Plasmodium falciparum is another single celled protozoan parasite whose life cycle also alternates between growth in an insect vector (mosquitoes) and a mammalian host. P. falciparum and other Plasmodium species (P. vivax, P. malariae, and P. ovale) are intracellular parasites that reside initially in liver parenchymal cells and then in red blood cells of the infected mammalian host where they cause the disease malaria. These Plasmodium species contain many genes that are expressed predominately at the intracellular stages in liver cells and/or red blood cells, and are either not expressed or expressed at very low levels in the insect stages or in cultured media. A head-to-head two-promoter is generated containing all, or part, of one or more Plasmiodium genes expressed predominately at the one or more of the intracellular stages of the parasite. Thus, the dsRNA encoded on the two-promoter plasmid is directed against one or more of the mRNAs from the Plasmodia genes that are predominately active during the intracellular stages, thereby killing the intracellular form of the parasite. The engineered Plasmodium is useful as an attentuated, live vaccine.

All publications, patents and patent documents are incorporated by reference herein, as though individually incorporated by reference. The invention has been described with reference to various specific and preferred embodiments and techniques. However, it should be understood that many variations and modifications may be made while remaining within the scope of the invention.

What is claimed is:

1. A eukaryotic double-stranded RNA (dsRNA) expression vector effective in a eukaryotic cell comprising:
(a) a designated DNA sequence of interest; and
(b) a pair of promoters on opposite ends of the designated DNA, wherein the promoters are oriented towards each other and wherein each is capable of transcribing a strand of DNA into RNA.

2. The dsRNA expression vector of claim 1, wherein the eukaryotic cell is a protist cell.

3. The dsRNA expression vector of claim 2, wherein the protist cell is a protozoan parasite cell.

4. The dsRNA expression vector of claim 2, wherein the protist cell is a Trypanosoma, Leishmania, Toxoplasma, or Plasmodium cell.

5. The dsRNA expression vector of claim 1, wherein the designated DNA sequence of interest is a random DNA sequence.

6. The dsRNA expression vector of claim 1, wherein the designated DNA sequence is a known DNA sequence.

7. The dsRNA expression vector of claim 1, wherein the designated DNA sequence is an essential gene from a protist.

8. The dsRNA expression vector of claim 1, wherein the designated DNA sequence is an essential gene from a protozoan parasite.

9. The dsRNA expression vector of claim 8, wherein the designated DNA sequence is an essential gene from Trypanosoma, Leishmania, Toxoplasma, or Plasmodia.

10. The dsRNA expression vector of claim 8, wherein the designated DNA sequence is an gene that is normally active during the protozoan's lifecycle when the protozoan is living in a mammalian host.

11. The dsRNA expression vector of claim 8, wherein the designated DNA sequence is an gene that is normally active during the protozoan's lifecycle when the protozoan is living in a mammalian host, but is not active during the protozoan's lifecycle when the protozoan is living in an insect host.

12. The dsRNA expression vector of claim 1, wherein each of the pair of promoters is the same type of promoter.

13. The dsRNA expression vector of claim 1, wherein each of the pair of promoters is a different type of promoter.

14. The dsRNA expression vector of claim 1, wherein at least one of the pair of promoters is a eukaryotic, prokaryotic or viral promoter.

15. The dsRNA expression vector of claim 14, wherein at least one of the pair of promoters is a ribosomal RNA promoter, a T. brucei variant surface glycoprotein (VSG) gene promoter, or a procyclic acidic repetitive protein (PARP) gene promoter.

16. The dsRNA expression vector of claim 1, wherein at least one of the pair of promoters is a bacteriophage T7 promoter, a bacteriophage T3 promoter, or an bacteriophage SP6 promoter.

17. The dsRNA expression vector of claim 1, wherein at least one of the pair of promoters is an inducible promoter.

18. The dsRNA expression vector of claim 17, wherein the ribosomal RNA promoter is derived from Trypanosoma, Leishmania, Toxoplasma, or Plasmodia.

19. The dsRNA expression vector of claim 1, further comprising (c) a vector backbone.

20. The dsRNA expression vector of claim 19, wherein the backbone is a Trypanosoma, a Leishmania, a Toxoplasma, or a Plasmodia expression vector.

21. The dsRNA expression vector of claim 1, wherein the vector is effective in a protist.

22. The dsRNA expression vector of claim 21, wherein the protist is Trypanosoma, Leishmania, Toxoplasma, or Plasmodia.

23. The dsRNA expression vector of claim 1, further comprising sequences for integrating the vector into the genome of the eukaryotic cell.

24. The dsRNA expression vector of claim 1, further comprising an expression regulatory region.

25. The dsRNA expression vector of claim 24, wherein the regulatory region comprises a tetracycline operator sequence, a lactose operator sequence, a transcription termination sequence or another transcription regulatory element.

26. A eukaryotic cell containing a eukaryotic double-stranded RNA (dsRNA) expression vector comprising:
(a) a designated DNA sequence of interest; and
(b) a pair of promoters on opposite ends of the designated DNA, wherein the promoters are oriented towards each other and wherein each is capable of transcribing a strand of DNA into RNA.

27. The eukaryotic cell of claim 26, wherein the vector is integrated into the genome of the eukaryotic cell.

28. The eukaryotic cell of claim 26, further comprising an expression regulatory region.

29. The eukaryotic cell of claim 26, wherein at least one of the pair of promoters is an inducible promoter.

30. A vaccine comprising a eukaryotic cell containing, in combination with a physiologically-acceptable, non-toxic vehicle, a eukaryotic double-stranded RNA (dsRNA) expression vector comprising a designated DNA sequence of interest and a pair of promoters on opposite ends of the designated DNA, wherein the promoters are oriented towards each other and wherein each is capable of transcribing a strand of DNA into RNA.

31. The vaccine of claim 30, wherein the vaccine further comprises an immunological adjuvant.

32. The vaccine of claim 30, wherein the eukaryotic cell is a protist cell.

33. The vaccine of claim 32, wherein the protist cell is a protozoan parasite cell.

34. The vaccine of claim 33, wherein the protist cell is a Trypanosoma, Leishmanria, Toxoplasma, or Plasmodium cell.

35. The vaccine of claim 30, wherein the vector is integrated into the genome of the eukaryotic cell.

36. The vaccine of claim 30, farther comprising an expression regulatory region.

37. The vaccine of claim 30, at least one of the pair of promoters is an inducible promoter.

38. A method of protecting a susceptible mammal against colonization or infection of a eukaryotic pathogen comprised of administering to the mammal an effective amount of a vaccine comprising a eukaryotic pathogenic cell, wherein the cell contains, in combination with a physiologically-acceptable, non-toxic vehicle, a eukaryotic double-stranded RNA (dsRNA) expression vector comprising a designated DNA sequence of interest and a pair of promoters on opposite ends of the designated DNA, wherein the promoters are oriented towards each other and wherein each is capable of transcribing a strand of DNA into RNA.

39. The method of claim 38, wherein the vaccine further comprises an immunological adjuvant.

40. The method of claim 38, wherein the vaccine is administered by subcutaneous or intramuscular injection.

41. The method of claim 38, wherein the vaccine is administered by oral ingestion.

42. The method of claim 38, wherein said vaccine is administered intranasally.

43. The metho d of claim 38, wherein the protist is a protozoan parasite.

44. The method of claim 43, wherein the protist is Trypanosoma, Leishmania, Toxoplasma, or Plasmodia.

45. The method of claim 38, wherein the mammal is a human, dog, bovine, porcine, or equine.

46. The method of claim 45, wherein the mammal is a human.

47. The method of claim 38, wherein the vector is integrated into the genome of the eukaryotic cell.

48. The method of claim 38, further comprising an expression regulatory region.

49. The method of claim 38, at least one of the pair of promoters is an inducible promoter.

50. A method of generating double-stranded RNA (dSRNA) comprising culturing a eukaryotic cell that contains a eukaryotic double-stranded RNA (dsRNA) expression vector comprising a designated DNA sequence of interest; and a pair of promoters on opposite ends of the designated DNA, wherein the promoters are oriented towards each other and wherein each is capable of transcribing a strand of DNA into RNA.

51. The method of claim 50, wherein the eukaryotic cell is a protist cell.

52. The method of claim 51, wherein the protist cell is a protozoan parasite cell.

53. The method of claim 52, wherein the protist cell is a Trypanosoma, Leishmania, Toxoplasma, or Plasmodium cell.

54. The method of claim 50, further comprising administering a compound so as to regulate transcription of dsRNA by the expression vector.

55. The method of claim 54, wherein the compound stimulates synthesis of dsRNA by the expression vector.

56. The method of claim 54, wherein the compound inhibits synthesis of dsRNA by the expression vector.

57. The method of claim 54, wherein the vector further comprises an expression regulatory region.

58. The method of claim 54, at least one of the pair of promoters is an inducible promoter.

59. A method of screening designated nucleic acids capable of inhibiting expression of an essential eukaryotic gene comprising introducing a eukaryotic double-stranded RNA (dsRNA) expression vector into a eukaryotic cell, wherein the expression vector comprises a designated DNA sequence of interest and a pair of promoters on opposite ends of the designated DNA, wherein the promoters are oriented towards each other and wherein the expression vector produces dsRNA, and measuring the ability of the dsRNA to inhibit expression of the corresponding endogenous DNA.

60. The method of claim 59, wherein the eukaryotic cell is a protist cell.

61. The method of claim 60, wherein the protist cell is a protozoan parasite cell.

62. The method of claim 61, wherein the protist cell is a Trypanosoma, Leishmania, Toxoplasma, or Plasmodium cell.

63. The method of claim 59, wherein the expression vector further comprises sequences for integrating the vector into the genome of the eukaryotic cell.

64. The method of claim 59, wherein the expression vector further comprises an expression regulatory region.

65. The method of claim 59, at least one of the pair of promoters is an inducible promoter.

66. The method of claim 59, further comprising administering a compound so as to regulate transcription of dsRNA by the expression vector.

67. The method of claim 66, wherein the compound stimulates synthesis of dsRNA by the expression vector.

68. The method of claim 67, wherein the compound inhibits synthesis of dsRNA by the expression vector.
